(19) United States
(12) Patent Application Publication
(10) Pub. No.: US 2004/0014218 A1
(43) Pub. Date: Jan. 22, 2004

(54) Dhfr-deficient cho cell line transfected with an anti-apoptotic gene, method for preparation thereof, and method for producing target protein using the same

(76) Inventors: Gyun Min Lee, Taejon (KR); Suk Kyoo Lee, Kyunggi-do (KR)

Correspondence Address: MUSERLIAN AND LUCAS AND MERCANTI, LLP, 475 PARK AVENUE SOUTH, NEW YORK, NY, 10016, US

(21) Appl. No.: 10/380,809

(22) PCT Filed: Jun. 12, 2002
(86) PCT No.: PCT/KR02/01113

(51) Int. Cl. C12N 15/861; C07K 14/71; C12N 5/06

(52) U.S. Cl. 435/455; 435/358; 435/456; 530/350

(57) ABSTRACT

A DHFR-deficient CHO cell line is transfected with anti-apoptotic gene, a method prepares the DHFR-deficient CHO cell line, and a method produces target proteins using the DHFR-deficient CHO cell line. Protein production using animal cells is limited by the low productivity of animal cells compared to microbial cells. Therefore, inhibition of apoptosis is expected to increase in productivity of target proteins by extending longevity of the transfected CHO cell line and to maintain the molecular integrity of unstable target proteins in a medium by decreasing cell lysis.

### FIELD OF THE INVENTION

The present invention relates to a DHFR-deficient CHO cell line transfected with an anti-apoptotic gene, a method for preparation thereof, and a method for producing target proteins using the same.

### BACKGROUND OF THE INVENTION

In the field of biology and medical science, a desired target protein can be obtained mainly by culturing transfected cell lines. The methods using CHO dhfr(-), CHO K1, BHK cell line, and NSO are examples used for the production of target proteins in the industry (Ogata, et al., Applied Microbiology and Biotechnology, 1993, 38(4), 520-525; Kratje, et al., Biotechnology Progress, 1994, 10(4), 410-20; Peakman, et al., Human Antibodies Hybridomas, 1994, 5(1-2), 65-74).

Among the above cell lines, the DHFR-deficient CHO cell line is the most commonly used host cell line for the mass-production of target proteins using animal cells in the industry. There are five main reasons that the DHFR-deficient CHO cell line is industrially preferred:

(1) The posttranslational modification process of protein, that is, glycosylation or phosphorylation process, is similar to that of the human cells.

(2) Suspension culturing as well as adhesion culturing of the cell is possible.

(3) Relatively high concentrations of cells can be achieved compared with other cell lines cultured in a serum-free culture medium.

(4) The productivity of the target protein, which is significantly lower than that of other microorganisms, can be increased by the dihydrofolate reductase/methotrexate (DHFR/MTX) amplifying system.

(5) Since safety and stability of the DHFR-deficient CHO cell line has been verified, the cell line can be easily approved by supervisory institutions such as the FDA.

Recombinant CHO cell lines producing target proteins are produced by transfecting a target gene into the DHFR-deficient CHO cell line. To mass-produce target proteins industrially using a recombinant CHO cell line, the recombinant CHO cells should be cultured as suspended forms in a serum-free culture media. Since many unidentified proteins are present in the serum, the serum should be excluded throughout the entire culturing process. By doing so, the expense and effort required for the subsequent purification process can be spared. In addition, recently, supervisory institutions such as the FDA require the exclusion of serum throughout the entire process due to an outbreak of mad cow disease. When the CHO cell line is cultured as a suspended form in a serum-free culture media, however, the amount of produced target protein tends to decrease due to the apoptosis caused by programmed cell death (Itoh, et al., Biotechnology and Bioengineering, 1995, 48, 118-122; Suzuki, et al., Cytotechnology, 1997, 23, 55-59; Simpson, et al., Biotechnology and Bioengineering, 1997, 54, 1-16). Furthermore, the decrease of survival rate caused by programmed cell death not only decreases the productivity of target proteins but also affects the stability of target proteins when various proteases, present inside the cells, are secreted as the cells underwent lysis. Thus, the DNA and cell debris of the lysed cells complicates the subsequent purifying process. In addition, when sodium butyrate (NaBu) is added in order to increase the amount of target proteins, apoptosis caused by programmed cell death tends to be increased.

The mechanism of programmed cell death is as follows.

When the initiator caspase, a kind of protease, is activated by various stimuli, the membrane potential of mitochondria is disintegrated. Thereafter, cytochrome C, which is involved in the electron transfer system of mitochondria, is released from the cytoplasm. Cytochrome C released into the cytoplasm activates the effector caspase such as caspase 3, and thus, phophatidylserine, one of the main components of the phospholipid in the cell membrane, flips towards the cytoplasm. Accordingly, the DNA is digested by the activated endonuclease, and thus, the cell eventually undergoes apoptosis.

Meanwhile, Bcl-2 and adenovirus-derived E1B-19K proteins inhibit the caspase activity around the mitochondrial membrane, resulting in the inhibition of apoptosis caused by programmed cell death (Desagher, et al., Trends in Cell Biology, 2000, 10, 369-376; Reed, et al., Biochemica et Biophysica Acta, 1998, 1366, 127-137; Tsujimoto, et al., FEBS Letters, 2000, 466, 6-10; Li, et al., Current Opinion in Cell Biology, 1999, 11, 261-266).

Therefore, the present inventors prepared CHO dhfr(-) cell lines transfected with a gene coding for the anti-apoptotic protein and completed the present invention showing that apoptosis can be decreased and target protein can be mass produced using the transfected cell line.

### SUMMARY OF THE INVENTION

It is an object of the present invention to provide a DHFR-deficient CHO cell line transfected with an anti-apoptotic gene, a method for preparation thereof, and a method for producing target protein using the same.

### BRIEF DESCRIPTION OF THE DRAWINGS

FIG. 1A is a schematic diagram of the gene map of a vector comprising Bcl-2 gene (pIRES-neo-Bcl2).

FIG. 1B is a schematic diagram of the gene map of a vector comprising adenovirus derived E1B-19K gene (pCDNA3.1-zeo-E1B).

FIG. 2A is a Western blot analysis showing that Bcl-2 protein is overexpressed in the DHFR-deficient CHO cells introduced by Bcl-2 gene.

FIG. 2B is a Western blot analysis showing that E1B-19K protein is overexpressed in the DHFR-deficient CHO cell introduced by adenovirus-derived E1B-19K gene.

FIG. 3A is a graph comparing cell concentration and cell survival rate of the DHFR-deficient CHO cell line overexpressing Bcl-2 protein with those of a control group of DHFR-deficient CHO cells cultured as a batch-type suspension culture in a serum-free culture medium.

&Rectversolid;; the amount of cells (Bcl-2 transfected)

&Ovalsolid;; the amount of cells (control)

[symbol] ; the survival rate (Bcl-2 transfected)

&Ovalhollow;; the survival rate (control)

FIG. 3B is a graph comparing cell concentration and cell survival rate of the DHFR-deficient CHO cell line overexpressing adenovirus-derived E1B-19K protein with those of a control group of DHFR-deficient CHO cells cultured as a batch-type suspension culture in a serum-free culture medium.

▴; the amount of cells (E1B transfected)

&Ovalsolid;; the amount of cells (control)

Δ; the survival rate (E1B transfected)

&Ovalhollow;; the survival rate (control)

FIG. 4A is a graph showing the fraction of dying cells by programmed cell death by staining the cells with mixed solution of acridine orange and ethidium bromide, when the DHFR-deficient CHO cell line overexpressing Bcl-2 protein is batch-type cultured.

&Rectversolid;; CHO dhfr(-) Bcl2 NVA

&Ovalsolid;; CHO dhfr(-) control group NVA

[symbol] ; CHO dhfr(-) control group NVN

&Ovalhollow;; CHO dhfr(-) Bcl2 NVN

FIG. 4B is a graph showing the fraction of dying cells by programmed cell death by staining the cells with mixed solution of acridine orange and ethidium bromide, when the DHFR-deficient CHO cell line overexpressing adenovirus-derived E1B-19K protein is batch-type cultured.

▴; CHO dhfr(-) control group NVA

&Ovalsolid;; CHO dhfr(-) E1B NVA

Δ; CHO dhfr(-) control group NVN

&Ovalhollow;; CHO dhfr(-) E1B NVN

FIG. 5 is a graph showing the increase of specific productivity of target protein by DHFR/MTX amplifying process in the DHFR-deficient CHO cells overexpressing Bcl-2 protein.

Bcl2-19; sample cell line

neo-04; control cell line

FIG. 6 is a set of graphs comparing cell survival rate and antibody productivity when CHO Bcl2-19-008 and CHO neo-04-008 cell lines were cultured as suspended forms in the serum-free culture media with those when sodium butyrate was added while being cultured as a batch type in the serum-free culture media.

▴; CHO neo-04-008 cells cultured as suspended form in serum-free culture media

Δ; CHO Bcl2-19-008 cells cultured as suspended form in serum-free culture media

563 ; CHO neo-04-008 cells cultured as batch type in serum-free culture media with 5 mM NaBu addition on day 3

[symbol] ; CHO Bcl2-19-008 cells cultured as batch type in serum-free culture media with 5 mM NaBu addition on day 3

FIG. 7 is a Western blot analysis showing that Bcl-2 protein is stably overexpressed after DHFR/MTX amplifying process in the DHFR-deficient and Bcl-2 overexpressing recombinant CHO cell line producing target protein.

### DETAILED DESCRIPTION OF PREFERRED EMBODIMENTS

Terminology and technology referenced in the present detailed description are used in conformance with the general meaning in the technical field to which the present invention applies. All references mentioned in the detailed description are for descriptive purposes only.

The method for preparing a DHFR-deficient CHO cell line transfected with an anti-apoptotic gene comprises steps of (1) adapting CHO dhfr(-) cell line, cultured as an attached form in the serum-supplemented culture medium, to the serum-free culture medium as a suspended form; (2) introducing a vector including the anti-apoptotic gene to the adapted CHO dhfr(-) cell line; and (3) selecting a CHO cell line overexpressing anti-apoptotic protein.

In step (1), the CHO dhfr(-) cell line is a cell line not expressing DHFR protein involved in hypoxanthine and thymidine synthesis, the essential components of the cell, and is commercially available (ATCC;CRL 9096). Further, the CHO dhfr(-) cell line can be prepared using a well-known transfection method by those skilled in the art. Examples of the transfection method include a transfection method induced by UV or by gamma irradiation. To prepare a cell line adapted to serum-free conditions, a series of culture media is used, wherein the serum concentration is gradually reduced and, ultimately, the culture medium is serum-free.

In step (2), anti-apoptotic genes are selectable from the group consisting of Bcl-2, adenovirus-derived E1B-19K, Bcl-X_{L}, Bcl-W, Mcl-1, and IAP. Nevertheless, the anti-apoptotic genes are not restricted to the above genes and any known anti-apoptotic gene can be used in the present invention. In a preferred embodiment of the present invention, Bcl-2 and adenovirus-derived E1B-19K were used.

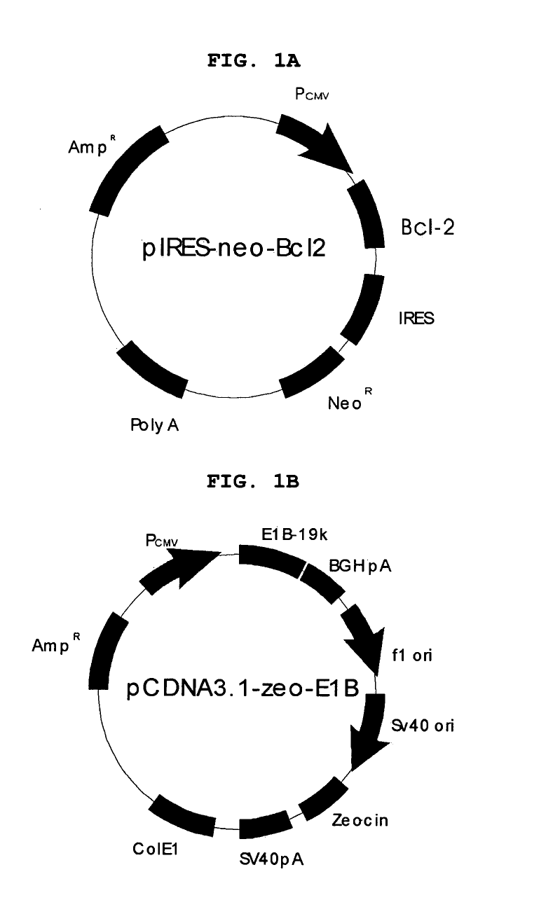

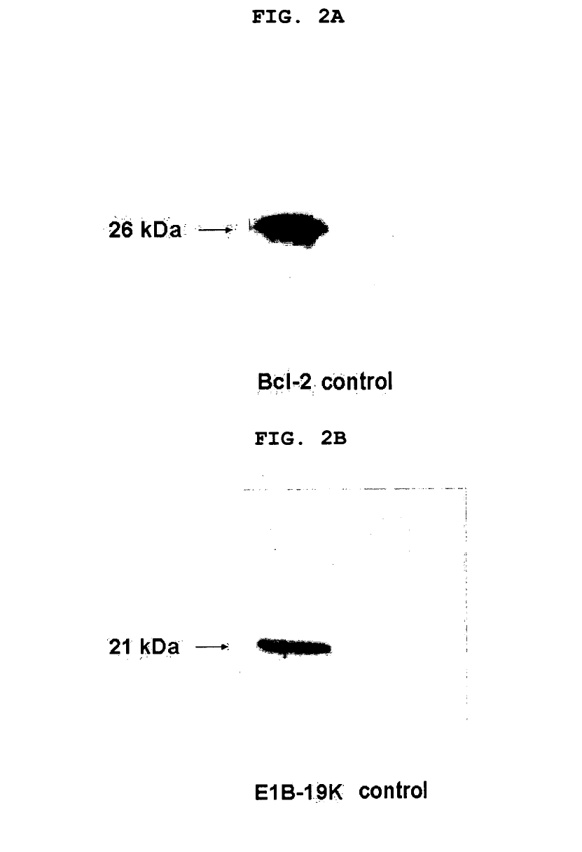

To prepare the DHFR-deficient CHO cell line transfected with an anti-apoptotic gene, the present inventors cultured a DHFR-deficient CHO host cell (ATCC: CRL 9096) as an attached form in an IMDM culture media supplemented with hypoxanthine, thymidine, and 10% fetal bovine serum (FBS). Suspension culturing of the attached cell line is performed in the mixed culture media of IMDM and CHO-S-SFM II culture media (Gibco, Grand Island, N.Y.) supplemented with 5% FBS (v/v). The DHFR-deficient CHO cells were continued to be subcultured in the same culture conditions when the cells reached the exponential growth phase. When cell growth rate was restored to the extent of the adhesion culture, suspension culture of the DHFR-deficient CHO cell line was performed in the mixed culture media of IMDM and CHO-S-SFMII media supplemented with hypoxanthine, thymidine, and 2.5% FBS (v/v). The DHFR-deficient CHO cells were continued to be subcultured in the same culture conditions when the cells reached to the exponential growth phase. Similarly, after the cells were adapted to the suspension culture in the mixed culture media of IMDM and CHO-S-SFMII media supplemented with hypoxanthine, thymidine and 1.25% FBS, respectively. Cell growth rate was restored to the extent of the adhesion culture, and the cells were cultured in the serum-free CHO-S-SFM II culture media supplemented with hypoxanthine and thymidine. After culturing DHFR-deficient CHO cell line adapted to the serum-free IMDM culture media supplemented with thymidine, and 10% FBS, Bcl-2 overexpression vector (pIRES-neo-Bcl2, see FIG. 1A) and adenovirus E1B-19K overexpression vector (pCDNA3.1-zeo-E1B, see FIG. 1B) is introduced to the cell line. After the vector was introduced, Bcl-2 overexpressing cell line was treated with G418 antibiotics and adenovirus-derived E1B-19K overexpressing cell line was treated with Zeocin antibiotics. Thus, cells showing resistance against each antibiotic were selected by culturing for two to three weeks. After cell selection, the present inventors selected cell lines overexpressing Bcl-2 and adenovirus E1B-19K protein using Western blot analysis.

The present inventors deposited the DHFR-deficient CHO cell line transfected with Bcl-2 gene and adenovirus-derived E1B-19K gene at the gene bank of Korea Research Institute of Bioscience and Biotechnology on Dec. 29, 2001 (deposit Nos. KCTC 10142Bp and KCTC 10143BP).

The present inventors investigated whether apoptosis caused by programmed cell death at the DHFR-deficient CHO cell line overexpressing Bcl-2 or adenovirus-derived E1B-19K was inhibited in the serum-free culture media when cultured as a suspended form. Here, the cell survival rate has been estimated after batch-type culturing of the control cell line and the transfected cell line of the present invention. In addition, after staining cells with mixed staining reagent of acridine orange and ethidium bromide, the amount of living cells and the amount of dead cells by programmed cell death or necrosis were estimated. The rate of nonviable apoptotic cells (NVA) and the rate of nonviable necrotic cells (NVN) among the total cells were also measured.

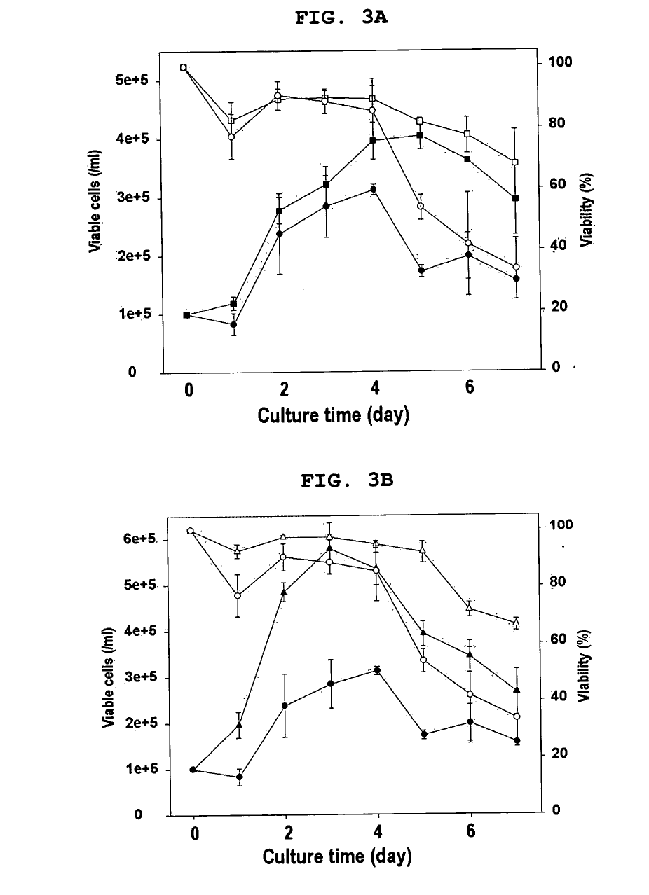

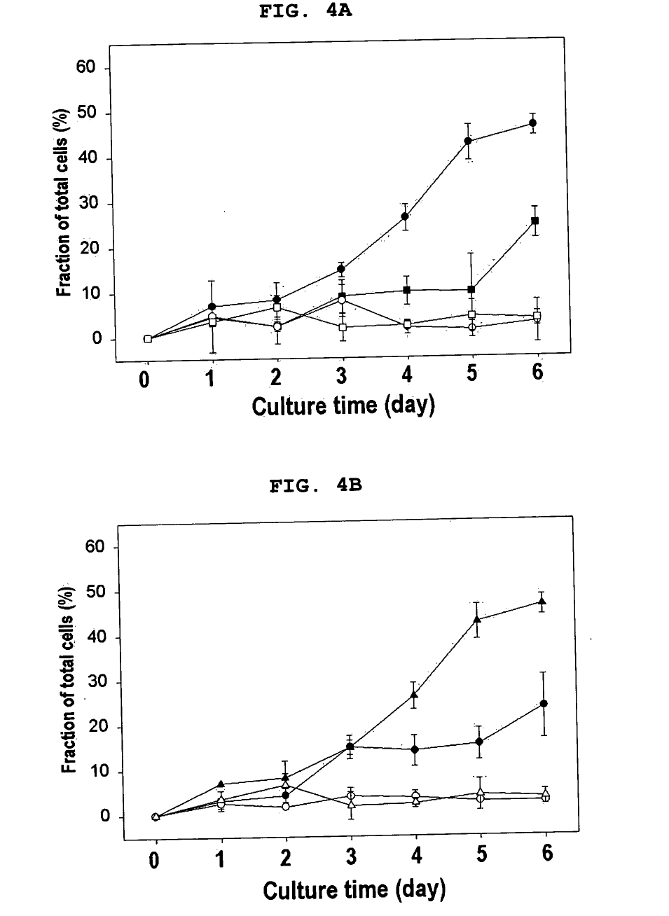

As a result, when cultured as a batch type in the serum-free culture media, it was found that cell survival rate of the control group rapidly decreased after exponential growth phase from one to two days, resulting about 50% of survival rate on the fifth day and about 30% of survival rate on the seventh day. Similarly, CHO dhfr(-) Bcl2 showed exponential growth phase from one to two days, whereas the cell line showed a cell survival rate of about 80% on the fifth day and about 30% on the seventh day of culturing (see FIGS. 3A and 3B). Even though the fractions of dying cells by programmed cell death show similar pattern at both cell lines until the third day of culturing, it has been found that the dying cells by programmed cell death form more than 40% of the control group on the sixth day and about 25% of the CHO dhfr(-) Bcl2 group (see FIGS. 4A and 4B). In conclusion, as for CHO dhfr(-) Bcl2, overexpression of Bcl-2 results in the extension of the cell survival by inhibiting the programmed cell death.

As a result of batch-type culturing of the CHO dhfr(-) E1B-19K cell line, the cell survival rate was maintained over 80% on the fifth day and about 60% on the seventh day, showing the extension of the cell survival rate compared with the control group. The dying fraction of the cells by programmed cell death among total cells is similar to that of the control group until the third day of culturing. On the sixth day of culturing, the fraction of dying cells of control group reached more than 40%, whereas that of the dhfr(-) E1B-19K cells reached about 25%. In conclusion, as for CHO dhfr(-) E1B-19K, overexpression of E1B-19K results in the extension of the cell survival rate by inhibiting the programmed cell death.

From the above results, it has been proved that CHO dhfr(-) Bcl2 cell line or CHO dhfr(-) E1B-19K cell line can be cultured as a suspension form in the serum-free culture media, and the cell survival rate of them can be extended by inhibiting programmed cell death in the serum-free culture media.

In addition, the present invention provides a method for production of target proteins using said transfected CHO cell line.

The method for producing target protein comprises steps of transfecting a vector comprising the gene for target protein into the transfected CHO cell line of the present invention and incubating the transfected CHO cell line.

The target protein is selectable from the group consisting of humanized antibodies, human interferon γ, factor VIII, erythropoietin, and thrombopoietin. Nevertheless, the target proteins are not restricted to this group only. As a preferred embodiment of the present invention, humanized antibody is used as a target protein.

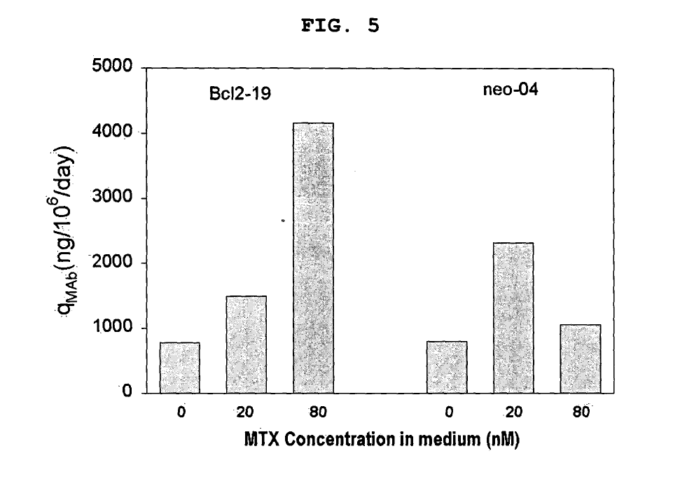

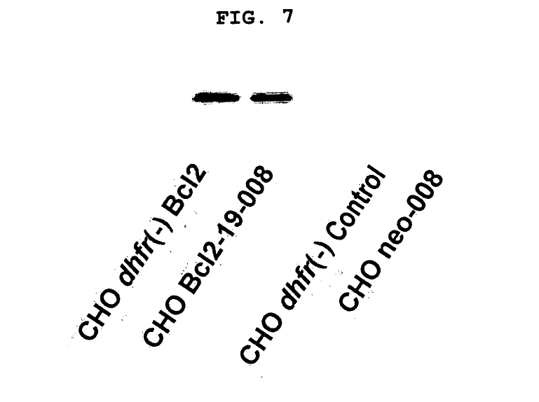

To determine whether a DHFR-deficient CHO cell line overexpressing an anti-apoptotic protein can be used to produce the target protein, the present inventors transfected the vector including the target gene into the DHFR-deficient CHO cell line after culturing the cell line in an IMDM culture medium supplemented with 10% FBS. Then, the transfected cells were selected by adding selective antibiotics into the IMDM culture media including 10% dialyzed FBS. After two to three weeks, transfected cells showing high productivity of target protein were selected among the selected cells. The cell lines established by the above method are called parental cell lines. After calculating the specific productivity of the parental cell line, cell lines showing increased specific productivity than that of the parental cell line were inoculated to the culturing plate. After amplification of the target gene for two to three weeks in the IMDM culture medium supplemented with 10% dialyzed FBS (v/v) and methotrexate, specific productivity was calculated through batch-type culturing (see FIG. 5). Using the above method, the specific productivity could be increased by elevating the concentration of MTX in the culture media. Since the cell line that goes through the gene amplifying process shows heterogeneity, a recombinant CHO cell line showing the highest specific productivity can be established from a limiting dilution process. As predicted, the recombinant CHO cell line producing target protein can be cultured as a suspension form in the serum-free culture media. Moreover, as confirmed by Western blot analysis, programmed cell death was inhibited by the overexpression of Bcl-2 (see FIG. 7).

The present invention is herein described with reference to the following examples.

### EXAMPLES

Practical and presently preferred embodiments of the present invention are illustrative as shown in the following Examples. It will be appreciated however, that those skilled in the art, on consideration of this disclosure, may make modifications and improvements within the spirit and scope of the present invention.

### Example 1

Preparation of DHFR-deficient CHO Cell Line Overexpressing Anti-Apoptotic Protein

To prepare a DHFR-deficient CHO cell line overexpressing an anti-apoptotic protein, the present inventors first cultured the DHFR-deficient CHO DUKX cell line (ATCC: CRL 9096) as an attached form in an IMDM culture media (Gibco, Grand Island, N.Y.) supplemented with 100 μm of hypoxanthine, 16 μm of thymidine, and 10% FBS. Then, the attached cells were inoculated into 50 ml of culture media prepared by mixing the IMDM media (supplemented with 100 μm of hypoxanthine, 16 μm of thymidine, and 5% FBS) and CHO-S-SFMII (Gibco, Grand Island, N.Y.) to a 1:1 volume ratio and were then cultured as a suspended form in the spinner flask. When the cell reached exponential growth phase three to four days later, the cell kept subculturing at the same culturing conditions as above. When cell growth rate was restored to the extent of the adhesion culture, the attached cells were inoculated into the 50 ml of culture media prepared by mixing the IMDM media supplemented with 100 μM of hypoxanthine, 16 μM of thymidine, and 2.5% FBS (v/v), and CHO-S-SFMII to a 1:1 volume ratio and cultured as a suspended form in the spinner flask. When the cell reaches exponential growth phase three to four days later, the cells were kept subculturing at the same culturing conditions as above. Similarly, after the cells were adapted to the suspension culture in the mixed culture media of IMDM and media supplemented with 100 μM of hypoxanthine, 16 μM of thymidine, and 1.25% FBS, and cell growth rate is restored to the extent of the adhesion culture, the cells were cultured as a suspended form in the serum-free CHO-S-SFM II culture media supplemented with 100 μM of hypoxanthine and 16 μM of thymidine.

The DHFR-deficient CHO DUKX cell line which was adapted in the suspension culturing was then inoculated to the concentration of 10⁵ cells/ml in 5 ml of IMDM culture media supplemented with 100 μM of hypoxanthine, 16 μM of thymidine, and 10% FBS (v/v). After culturing for 24 hours, Bcl-2 overexpression vector (pIRES-neo-Bcl2, FIG. 1A) and adenovirus E1B-19K overexpression vector (pCDNA3.1-zeo-E1B, FIG. 1B), which was prepared by pIRES-neo vector (Invitrogen) and pCDNA3.1-zeo vector (Clontech), was introduced respectively to the cell line by liposome method. After 48 hours, Bcl-2 overexpressing cell line was treated with 550 μg/ml concentrations of G418 antibiotics (Gibco) and adenovirus-derived E1B-19K overexpressing cell line was treated with 550 μg/ml concentrations of Zeocin antibiotics (Invitrogen). By culturing the cells in a 96-well plate for two to three weeks, the cells showing resistance against each antibiotic were selected. After that, the present inventors selected cell lines overexpressing Bcl-2 and adenovirus-derived E1B-19K protein using Western blot analysis. The control cells were cells introduced with the vector not containing the target gene and selected in the selective culture media containing antibiotics.

More specifically, after incubating 10⁷ cells of the CHO dhfr(-) Bcl-2 cell line and a control cell line on the exponential growth phase in 1 ml of lysis buffer containing 1% NP-40, 0.1% sodium dodecylsulfate (SDS), 0.02% NaN₃, 50 mM Tris (pH8.0), 150 mM NaCl, 100 mg/ml phenylmethanesulfonylfluoride (PMSF), and 1 μg/ml aprotinin for thirty minutes at 4° C., the present inventors obtained a supernatant by centrifuging the cells to 16,000 g for five minutes at 4° C. After electrophoresis of each supernatant in 15% SDS PAGE gel, the proteins were transferred to the hybond-enhanced chemiluminescence nitrocellulose (Amersham Pharmacia Biotech, Piscataway, N.J.) for twelve hours at 40V. After blocking the transferred proteins on the nitrocellulose membrane for one hour in 5% nonfat milk, the proteins were visualized by an ECL Western blot system (Amersham Pharmacia Biotech) using mouse anti-human Bcl-2 monoclonal antibody (Sigma) as a primary antibody and goat anti-mouse IgG polyclonal antibody (Upstate Biotechnology, Lake Placid, N.Y.) combined with horseradish peroxidase as a secondary antibody. Similarly, the CHO dhfr(-) E1B-19K cell line was visualized by Western blot analysis.

As a result, the CHO dhfr(-) Bcl-2 cell line stably expressed Bcl-2 protein of 26 kDa in size, whereas the control group did not express Bcl-2. The reason is because, even though Bcl-2 is present in the CHO cell line itself, the overexpressed Bcl-2 was of human-origin and an antibody specific for human Bcl-2 was used as an primary antibody. In addition, the CHO dhfr(-) cell line stably expressed adenovirus derived E1B-19K protein of 21 kDa in size, whereas the control group did not express any adenovirus-derived E1B-19K protein whatsoever.

The present inventors deposited the DHFR-deficient CHO cell line transfected with Bcl-2 gene and adenovirus-derived E1B-19K gene at the gene bank of Korea Research Institute of Bioscience and Biotechnology on Dec. 29, 2001 (deposit Nos. KCTC 10142BP and KCTC 10143BP).

### Example 2

Measurement of Apoptosis Inhibition Activity in the DHFR-Deficient CHO Cell Line Overexpressing Anti-Apoptotic Protein

To investigate whether the apoptosis, caused by programmed cell death in the DHFR-deficient CHO cell line overexpressing Bcl-2 gene (CHO dhfr(-) Bcl2) and in the DHFR-deficient CHO cell line overexpressing adenovirus-derived E1B-19K gene (CHO dhfr(-) E1B-19K), was inhibited when cultured as a suspended form in serum-free culture media, the present inventors conducted the following experiment.

After inoculating the CHO dhfr(-) Bcl2 and CHO dhfr(-) E1B-19K cell line to the initial concentrations of 10⁵ cells/ml into 50 ml of serum-free CHO-S-SFMII media supplemented with 100 μm of hypoxanthine and 16 μm of thymidine, the cell lines were batch-type cultured in the spinner flask. Similarly, the control cell line was batch-type cultured under the same culturing conditions as above. Every 24 hours after being inoculated, the cells in the 1 ml of culture media were selected and stained with trypan blue to distinguish dead and living cells using hemacytometer, to thus estimate the cell survival rate. In addition, after staining the cells with mixed staining reagent of 1.5 μg/ml of acridine orange and 7.5 μg/ml of ethidium bromide, the amount of living cells and the amount of dead cells due to programmed cell death or necrosis were estimated using an epifluorescence microscope (Nikon Microphot-FXA). Furthermore, the ratio of nonviable apoptotic cells (NVA) and nonviable necrotic cells (NVN) among total cells was measured.

As a result, when cultured as batch type in the serum-free culture media, the cells of the control group were under exponential growth phase from one to two days. Thereafter, the cell survival rate of the control group rapidly decreased, with about 50% of the cells dying on the fifth day of culturing and 70% dying on the seventh day. By contrast, CHO dhfr(-) Bcl2cells also were found to be under exponential growth phase from one to two days after inoculation, but the cell survival rate of the CHO dhfr(-) Bcl2 cells was about 80% on the fifth day and 70% on the seventh day (FIGS. 3A and 3B). Even though the fraction of dying cells by programmed cell death showed similar patterns at both of the cell lines until the third day of culturing, the percentage of the dying cells by programmed cell death formed more than 40% of the control group on the sixth day of culturing. The percentage of the CHO dhfr(-) Bcl2 cells, however, was about 25% on the sixth day (FIGS. 4A and 4B). In conclusion, as for CHO dhfr(-) Bcl2, the cell survival rate was extended by inhibition of the programmed cell death through the overexpression of Bcl2 protein.

When CHO dhfr(-) E1B-19K cells were cultured as batch type, the cell survival rate was over 80% until the fifth day of culturing and was about 60% on the seventh day, showing an extended cell survival rate over that of the control group. Even though the ratio of dying cells by programmed cell death showed similar patterns for both the control and CHO dhfr(-) E1B-19K cell lines until the third day of culturing, the percentage of the dying cells by programmed cell death formed more than 40% of the control group on the sixth day of culturing. The percentage of the CHO dhfr(-) E1B-19K cells was about 25% on the sixth day. In conclusion, as for CHO dhfr(-) E1B-19K, the cell survival rate was extended by inhibition of the programmed cell death through the overexpression of E1B-19K protein.

From the above results, it is confirmed that CHO dhfr(-) Bcl2 cell line or CHO dhfr(-) E1B-19K cell line can be cultured as a suspended form in the serum-free media, and the cell survival rate can be extended by inhibition of the programmed cell death in the serum-free media.

### Example 3

Production of Target Proteins Using DHFR-Deficient CHO Cell Line Overexpressing Anti-Apoptotic Protein

To investigate whether the DHFR-deficient CHO cell line overexpressing the anti-apoptotic protein can be used for the production of target proteins, the present inventors performed the following experiment.

First, the cell was inoculated to a 60 mm culture dish in 5 ml of IMDM culture media supplemented with 100 μm of hypoxanthine, 16 μm of thymidine, and 10% FBS. After incubation for 24 hours, the vector comprising the gene for the target protein was transfected again to the cell line transfected with anti-apoptotic gene prepared in Example 1, using a liposome method. Exactly 48 hours later, the transfected cell line was selected by adding selective antibiotics to the IMDM culture media. In this case, the IMDM culture media was not supplemented with hypowanthine/thymidine but with 10% dialyzed FBS. After two to three weeks, the selected cells were divided duplicate in the 96-well plate, and then the amount of living cells was estimated by MTT analysis. Meanwhile, after collecting the culture media, an ELISA assay for the target protein was performed. The cell line showing a high productivity/cell rate was selected using both ELISA and MTT assay. The cell line thus established was designated the parental cell line, and the specific productivity of the parental cell line was calculated.

In more detail, after inoculating the parental cell line to the initial concentrations of 10⁵ cells/ml in a 10 mm culture dish, the gene for target protein was amplified for two to three weeks in an IMDM culture media supplemented with 20 nM of methotrexate and 10% dialyzed FBS. After the lapse of about two to three weeks, the specific productivity of the selected cells was measured after two to three weeks. After inoculating the cell line showing increased specific productivity compared with that of the parental cell line to the concentrations of 10⁵ cells/ml in a 100 mm culture dish, the gene for target protein was amplified for two to three weeks in the IMDM culture media supplemented with 80 nM of MTX and 10% dialyzed FBS. Then, the specific productivity was measured again after gene amplification by batch-type culturing (FIG. 5). Similarly, the specific productivity can be increased by gene amplification when the concentration of MTX in the culture media was increased to 320 nM to 1 μM. Since the cell line undergoing the gene amplification process showed heterogeneity, a CHO cell line with the highest specific productivity could be selected through the limiting dilution of the cells up to 0.4 to 0.8 cells per well in a 96-well plate.

To confirm the production of cell lines expressing target protein, the present inventors performed a batch-type suspension culture of the CHO Bcl2-19-008 and CHO dhfr(-) Bcl-2 cell lines producing humanized antibodies established from the CHO dhfr(-) Bcl-2 and the CHO dhfr(-) control cell lines, respectively, in the serum-free culture media. 0.5 ml of culture media was collected every day and the amount of living and dead cells was measured by the trypan blue exclusion method. The remaining culture media were used in the ELISA analysis to measure the amount of the produced humanized antibodies using goat anti-human IgG (Sigma) as coating antibodies and the goat anti-human IgG combined with peroxidase as enzyme-antibody conjugate. In addition, after adding 5 mM of sodium butyrate on the third day of culturing, which induces programmed cell death and acts as a transcription activator, the amount of produced antibodies was compared with that case of the batch type culture (Kim, et al., Biotechnology and Bioengineering, 2000/2001, 71, 184-193). Since the CHO Bcl2-19-008 and CHO neo-04-008 cell lines were established from two different host cell lines, the specific growth rate and specific productivity of antibody of the cell lines were different from each other. Therefore, the antibody productivity caused by the inhibition of programmed cell death was measured in both cases, i.e., when cells were cultured as a batch type in the serum-free culture media and when 5 mM of sodium butyrate was added to the serum-free culture media on the third day of culturing while being cultured as a batch type.

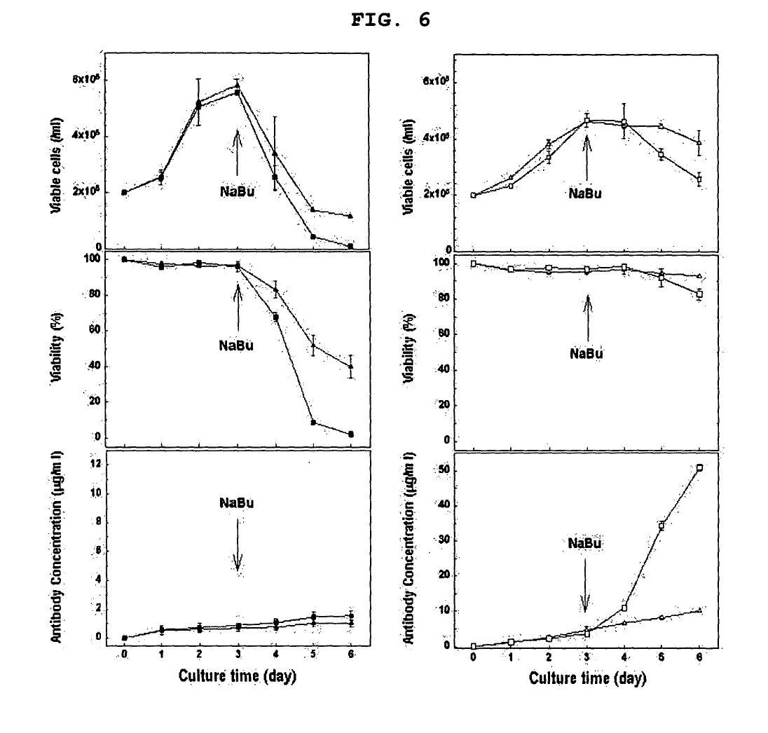

As a result, the amount of living cells in the CHO neo-04-008 cell line significantly decreased from the third day of culturing, and the cell survival rate also significantly decreased (FIG. 6). Cell survival rate was 40% on the sixth day of culturing, and the final antibody concentration was 1 μg/ml. When 5 mM of sodium butyrate was added, the cell survival rate decreased even more, and the cell survival rate was below 5% on the sixth day of culturing. When 5 mM of sodium butyrate was added, the final antibody concentration was increased to 1.5 μg/ml, which is about 1.5 times that of the suspension culture in the serum-free culture media. It is supposed that these results are due to the extent to which transcriptional activation was compensated with the rapid decrease of cell survival rate by the treatment of sodium butyrate. In contrast, CHO Bcl2-19-008 maintained a cell survival rate of over 90% until the sixth day of culturing, and the final antibody concentration was 10 μg/ml. Furthermore, when sodium butyrate was added, the cell survival rate was found to be about 80% on the sixth day of culturing, and the final antibody concentration was to 50 μg/ml, thus resulting in an increase of about five times that of the serum-free batch type culture.

From the above results, it was found that the recombinant CHO cell line of the present invention producing humanized antibody as a target protein could be cultured as a batch type. Furthermore, the overexpression of Bcl-2 inhibited programmed cell death (FIG. 7), and the increase of the final antibody concentration of CHO Bcl2-19-008 cell line resulted from the inhibition of programmed cell death by the overexpression of the Bcl-2 protein.

As described above, the transfected CHO cell line of the present invention exhibits excellent cell survival rate due to the inhibition of programmed cell death by the overexpression of the anti-apoptotic protein. Such extension of cell survival rate not only increases the productivity of target protein but also enhances the quality of protein produced by maintaining the integrity of the cell membrane, and thus, is useful to prepare the target protein.

Those skilled in the art will appreciate that the concepts and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

What is claimed is:

1. A DHFR-deficient CHO cell line transfected with an anti-apoptotic gene.

2. The transfected CHO cell line according to claim 1, wherein said anti-apoptotic gene is selected from the group consisting of Bcl-2, adenovirus-derived E1B-19K, Bcl-X_{L}, Bcl-W, Mcl-1, and IAP.

3. The transfected CHO cell line according to claim 1, wherein said anti-apoptotic gene is Bcl-2 (deposit No. KCTC 10142BP).

4. The transfected CHO cell line according to claim 1, wherein said anti-apoptotic gene is adenovirus derived E1B-19K (deposit No. KCTC 10143BP).

5. A method for preparing a DHFR-deficient CHO cell line transfected with an anti-apoptotic gene, comprising steps of:
adapting a CHO dhfr(-) cell line, cultured as an attached form in the serum-supplemented culture medium, to the serum-free culture medium as a suspended form
introducing a vector including the anti-apoptotic gene to the adapted CHO dhfr(-) cell line; and
selecting a CHO cell line overexpressing anti-apoptotic protein.

6. The method according to claim 5, wherein the anti-apoptotic gene is selected from the group consisting of Bcl-2, adenovirus-derived E1B-19K, Bcl-X_{L}, Bcl-W, Mcl-1, and IAP.

7. A method for producing target proteins, comprising steps of:
transfecting a vector including a gene for a target protein into a DHFR-deficient CHO cell line transfected with an anti-apoptotic gene; and
incubating the transfected CHO cell line.

8. The method according to claim 7, wherein the target protein is selected from the group consisting of humanized antibody, human interferon γ, factor VIII, erythropoietin, and thrombopoietin.
